(19) United States
(12) Patent Application Publication
(10) Pub. No.: US 2021/0177325 A1
(43) Pub. Date: Jun. 17, 2021

(54) INFORMATION PROCESSING APPARATUS AND NON-TRANSITORY COMPUTER READABLE MEDIUM

(71) Applicant: FUJI XEROX CO., LTD., Tokyo (JP)

(72) Inventors: Kengo TOKUCHI, Kanagawa (JP); Masahiro SATO, Kanagawa (JP)

(73) Assignee: FUJI XEROX CO., LTD., Tokyo (JP)

(21) Appl. No.: 16/864,572

(22) Filed: May 1, 2020

(30) Foreign Application Priority Data
Dec. 11, 2019 (JP) 2019-224004

(51) Int. Cl. A61B 5/16; A61B 5/0484

(52) U.S. Cl. CPC A61B 5/165; A61B 5/167; A61B 5/04842

(57) ABSTRACT

An information processing apparatus includes a processor configured to: obtain biological information of each of plural persons having a conversation; and, in a case where there is, among the plural persons, a specific person from whom biological information indicating a negative mental state is measured, control execution of a process of giving a change to at least one of the plural persons.

### CROSS-REFERENCE TO RELATED APPLICATIONS

This application is based on and claims priority under 35 USC 119 from Japanese Patent Application No. 2019-224004 filed Dec. 11, 2019.

### BACKGROUND

### (i) Technical Field

The present disclosure relates to an information processing apparatus and a non-transitory computer readable medium.

### (ii) Related Art

Japanese Unexamined Patent Application Publication No. 2009-082299 describes a mental-training system that allows a person to train his/her mental state on his/her own.

Japanese Unexamined Patent Application Publication No. 2015-109964 describes a feeling estimating apparatus that receives a subject's biological information other than brain waves, estimates the subject's brain waves from the biological information on the basis of the corresponding relationship between the biological information and brain waves, compares the estimated brain waves with a brain wave characteristic pattern stored in association with a feeling, and estimates the subject's feeling.

Japanese Unexamined Patent Application Publication No. 2019-095546 describes an interaction system that, in the case where the interaction with a user has been interrupted for a certain time period or longer, determines this as a conversation disrupted state, identifies the cause of the conversation disrupted state, determines the user's feeling, determines notification details for the user on the basis of a combination of the identified cause and the determined feeling, and notifies the user of the notification details.

### SUMMARY

By the way, people may sometimes be mentally hurt depending on the details of a conversation.

### SUMMARY

Aspects of non-limiting embodiments of the present disclosure relate to providing a mechanism for easing the mental pain of those who are presumed to be mentally hurt.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided an information processing apparatus including a processor configured to: obtain biological information of each of a plurality of persons having a conversation; and, in a case where there is, among the plurality of persons, a specific person from whom biological information indicating a negative mental state is measured, control execution of a process of giving a change to at least one of the plurality of persons.

### BRIEF DESCRIPTION OF THE DRAWINGS

An exemplary embodiment of the present disclosure will be described in detail based on the following figures, wherein:

FIG. 1 is a block diagram illustrating the configuration of an information processing system according to an exemplary embodiment;

FIG. 2 is a block diagram illustrating the configuration of an information processing apparatus according to the present exemplary embodiment;

FIG. 3 is a flowchart illustrating the flow of a process performed by the information processing apparatus according to the present exemplary embodiment;

FIG. 4 is a diagram illustrating a state management table;

FIG. 5 is a diagram illustrating a solution management table;

FIG. 6 is a graph illustrating changes in mental state over time;

FIG. 7 is a graph illustrating changes in mental state over time; and

FIG. 8 is a diagram that schematically illustrates persons, a robot, and so forth.

### DETAILED DESCRIPTION

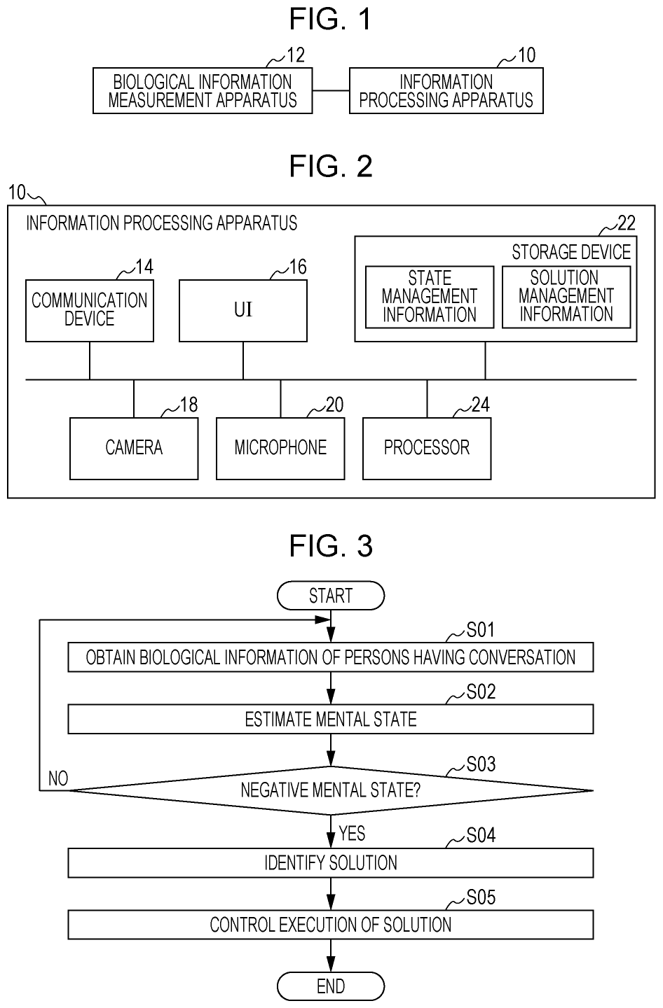

Referring to FIG. 1, an information processing system according to an exemplary embodiment will be described. FIG. 1 illustrates an example of the configuration of the information processing system according to the present exemplary embodiment.

The information processing system according to the present exemplary embodiment includes an information processing apparatus 10, and one or more biological information measurement apparatuses 12. The number of each type of apparatus illustrated in FIG. 1 is only exemplary. The information processing system may include other apparatuses (such as a terminal apparatus, a server, and other devices) other than the apparatuses illustrated in FIG. 1.

Each apparatus included in the information processing system is configured to communicate with another apparatus. The communication may be wired communication using a cable, or wireless communication. In short, each apparatus may be physically connected to another apparatus by a cable to transmit and receive information to and from each other, or to transmit and receive information to and from each other through wireless communication. As the wireless communication, short-range wireless communication, Wi-Fi (registered trademark), or the like used. Alternatively, wireless communication based on other standards may be used. Examples of short-range wireless communication include Bluetooth (registered trademark), radio frequency identifier (RFID), and near-field communication (NFC). Each apparatus may communicate with another apparatus via a communication channel such as a local area network (LAN) or the Internet.

The information processing apparatus 10 is, for example, a personal computer (hereinafter abbreviated as “PC”), a tablet PC, a smartphone, a cellular phone, or another apparatus (such as a server, a robot, or another device). The information processing apparatus 10 may be a terminal apparatus that may be carried by a person (such as a tablet PC, a smartphone, or a cellular phone), an apparatus installed and used on a table or the like, a movable apparatus (such as a self-propelled apparatus), an apparatus that performs a task, or an apparatus capable of interacting with a person. For example, the information processing apparatus 10 may be a smart speaker including a communication function, a microphone, and a speaker, or a robot that interacts with a person and performs a task. The information processing apparatus 10 may be an apparatus equipped with artificial intelligence (AI).

The biological information measurement apparatus 12 is an apparatus configured to measure a person's biological information. For example, the biological information measurement apparatus 12 has various sensors and electrodes, and measures a person's biological information. In the case where a plurality of biological information measurement apparatuses 12 are used, each biological information measurement apparatus 12 may measure a different type of biological information. One, some, or all of the biological information measurement apparatuses 12 may be configured to measure the same type of biological information. In addition, each biological information measurement apparatus 12 may be configured to measure one type of biological information, or may be configured to measure a plurality of types of biological information.

The biological information measurement apparatus 12 transmits measured biological information to the information processing apparatus 10. The biological information measurement apparatus 12 may transmit biological information to the information processing apparatus 10 whenever the biological information measurement apparatus 12 measures biological information, may store biological information and transmit the biological information to the information processing apparatus 10 at predetermined time intervals, or may transmit biological information to the information processing apparatus 10 at a time point specified by a person. In addition, the biological information measurement apparatus 12 may receive biological information measured by another biological information measurement apparatus 12 from this other biological information measurement apparatus 12, and may transmit biological information measured by the biological information measurement apparatus 12 and the biological information measured by this other biological information measurement apparatus 12 to the information processing apparatus 10.

The biological information measurement apparatus 12 may analyze biological information measured by the biological information measurement apparatus 12 or another biological information measurement apparatus 12, and transmit information indicating the analysis result to the information processing apparatus 10. The biological information measurement apparatus 12 includes a processor and a storage device, and the processor may analyze biological information. The analysis may be conducted by the information processing apparatus 10 or another apparatus.

The biological information measurement apparatus 12 may include a battery and be driven by power supplied from the battery, or may receive power supplied from another apparatus (such as the information processing apparatus 10) and be driven by the received power.

The biological information measurement apparatus 12 may be installed on a person. For example, the biological information measurement apparatus 12 may be a wearable apparatus that is worn by a person and that measures biological information. To give specific examples, the biological information measurement apparatus 12 may be an apparatus worn on a person's head (such as the forehead), a bearable apparatus worn on a person's ears, (such as an earphone or a headphone), an apparatus worn on a person's arm, hand, wrist, or finger (such as a watch-type apparatus including a smartwatch), an apparatus hung on a person's neck, an apparatus worn on a person's torso (such as the abdomen or chest), or an apparatus worn on a person's lower limb (such as a thigh, lower leg, knee, foot, or ankle). The biological information measurement apparatus 12 may be health equipment or the like worn on a person's arm, hand, torso, or lower limb. The biological information measurement apparatus 12 may be worn on other parts. In addition, a plurality of biological information measurement apparatuses 12 may be respectively worn on a plurality of parts.

Biological information includes various types of physiological information and anatomical information generated from a person who is a living body. The category of the concept of biological information includes, for example, information indicating brain activities (such as brain waves, brain blood flow, and brain magnetic field signals), information indicating a pulse rate, myoelectric information such as a myoelectric waveform, information regarding saliva (such as information indicating the volume of saliva), information indicating a pulse wave, information indicating a blood pressure, information indicating a blood flow, information indicating a pulse, information indicating a heart rate, information indicating an electrocardiographic waveform, information indicating eye movement, information indicating a body temperature, information indicating a sweat rate, information indicating the line of sight, audio information, and information indicating a person's movement. In addition, information identified by a biomarker may be used as biological information. These are merely examples of biological information, and other physiological information or anatomical information may be used as biological information. The biological information measurement apparatus 12 may measure one type or a plurality of types of these items of biological information.

In addition, the category of the concept of biological information includes biopotential information indicating a potential measured from a living body. The category of the concept of biopotential information includes brain waves, which are the result of measuring minute current generated by brain activity, an electrocardiogram, which is the result of measuring minute current generated by pulsation of the heart, an electromyogram, which is the result of measuring minute current generated by muscle activity, and a skin potential, which is the result of measuring minute current generated on the skin. These are merely examples of biopotential information, and other biopotential information may be used.

The information processing apparatus 10 receives biological information from the biological information measurement apparatus 12, analyzes the biological information, stores the biological information, outputs the biological information, stores information indicating the result of analysis of the biological information, and outputs information indicating the result of analysis of the biological information. Needless to say, the analysis of biological information may be conducted by the biological information measurement apparatus 12 or another apparatus. Outputting biological information includes, for example, displaying the biological information, and outputting the biological information as audio information. Outputting information indicating the result of analysis of biological information includes, for example, displaying information indicating the analysis result, and outputting the analysis result as audio information. The information processing apparatus 10 may transmit biological information and information indicating the analysis result to another apparatus.

The information processing apparatus 10 may include one or more biological information measurement apparatuses 12. In short, one or more biological information measurement apparatuses 12 may be incorporated in the information processing apparatus 10 to constitute one apparatus. The entire information processing apparatus 10 including one or more biological information measurement apparatuses 12 may be worn by a person and measure biological information. In short, the information processing apparatus 10 may be a wearable apparatus. For example, the information processing apparatus 10 may be an apparatus worn on a person's head (such as the forehead), a bearable apparatus worn on a person's ears, (such as an earphone or a headphone), an apparatus worn on a person's arm, hand, wrist, or finger (such as a watch-type apparatus including a smartwatch), an apparatus hung on a person's neck, an apparatus worn on a person's torso (such as the abdomen or chest), or an apparatus worn on a person's lower limb (such as a thigh, lower leg, knee, foot, or ankle). The information processing apparatus 10 may be health equipment or the like worn on a person's arm, hand, torso, or lower limb. The information processing apparatus 10 may be worn on other parts.

Needless to say, the information processing apparatus 10 and the biological information measurement apparatus 12 may be separate apparatuses. For example, the information processing apparatus 10 may be an apparatus such as a robot, a smart speaker, or a server, and the biological information measurement apparatus 12 may be a wearable apparatus worn by a person.

Hereinafter, the configuration of the information processing apparatus 10 will be described in detail with reference to FIG. 2. FIG. 2 illustrates an example of the configuration of the information processing apparatus 10.

The information processing apparatus 10 includes, for example, a communication device 14, a user interface (UI) 16, a camera 18, a microphone 20, a storage device 22, and a processor 24. The information processing apparatus 10 may include other configurations.

The communication device 14 is a communication interface, and has the function of transmitting data to another apparatus and the function of receiving data transmitted from another apparatus. The communication device 14 may have a wireless communication function or a wired communication function. The communication device 14 may communicate with another apparatus using, for example, short-range wireless communication, or may communicate with another apparatus via a communication channel such as a LAN or the Internet. The communication device 14 receives, for example, biological information transmitted from the biological information measurement apparatus 12. The communication device 14 may transmit control information for controlling the operation of the biological information measurement apparatus 12 to the biological information measurement apparatus 12.

The UI 16 is a user interface, and includes at least one of a display device and an operation device. The display device is, for example, a liquid crystal display or an electroluminescence (EL) display. The operation device includes, for example, a keyboard, input keys, and an operation panel. The UI 16 may be a UI such as a touchscreen that serves as both the display device and the operation device.

The camera 18 is an example of an image capturing device. Image data is generated by capturing an image with the camera 18. Image data may be still image data or moving image data. For example, an image of a person is captured with the camera 18. Image data may be analyzed by the processor 24 or another apparatus other than the information processing apparatus 10. Note that one or more cameras 18 are included in the information processing apparatus 10.

Sound data is generated by gathering sound with the microphone 20. The microphone 20 may be a sound collector that collects sound in a specific direction. For example, audio data representing a conversation held by people is generated by the microphone 20. Audio data may be analyzed by the processor 24 or another apparatus other than the information processing apparatus 10. Note that one or more microphones 20 are included in the information processing apparatus 10.

Note that the camera 18 and the microphone 20 may be provided outside the information processing apparatus 10, instead of being included in the information processing apparatus 10. In this case, the information processing apparatus 10 receives image data transmitted from the camera 18, and receives sound data transmitted from the microphone 20. Note that the information processing apparatus 10 may be provided with a speaker.

The storage device 22 is a device constituting one or more storage areas storing data. The storage device 22 includes, for example, a hard disk drive, various types of memory (such as random-access memory (RAM), dynamic RAM (DRAM), and read-only memory (ROM)), other storage devices (such as optical disks), or a combination thereof. One or more storage devices 22 are included in the information processing apparatus 10.

The storage device 22 stores state management information and solution management information.

State management information is information for estimating a person's state on the basis of the person's biological information. For example, predetermined reference biological information and state information indicating a person's state are linked in advance with each other and registered in the state management information. For each person, reference biological information and state information may be linked with each other and registered in the state management information.

Reference biological information is biological information estimated to be generated from a person having a state indicated by state information linked with the reference biological information. Reference biological information is determined by, for example, statistical processing, and is biological information that is estimated to be generally generated from a person having a state indicated by state information linked with the reference biological information.

For example, a person's state is the person's mental state. Reference biological information linked with state information indicating a person's mental state is said to be biological information indicating the person's mental state. The category of the concept of mental state includes the concept of feelings, moods, and the like.

Specifically, the category of the concept of mental state includes relief, anxiety, appreciation, shock, excitement, curiosity, calmness, emaciation, impatience, confusion, happiness, relaxation, stress, tension, responsibility, respect, affection, longing, motivation, fear, courage, pleasure, regret, satisfaction, dissatisfaction, resentment, disgust, shame, contempt, jealousy, guilt, expectation, superiority, inferiority, grudge, agony, sadness, emotion, anger, trouble, resignation, despair, hope, hatred, love, emptiness, favor, a feeling of wanting to escape, and refusal. Needless to say, the category of the concept of mental state may include other feelings, moods, and the like.

For example, mental states are classified as positive mental states and negative mental states.

For example, relief, appreciation, excitement, curiosity, calmness, happiness, relaxation, respect, affection, longing, motivation, courage, pleasure, satisfaction, expectation, superiority, emotion, hope, love, and favor are classified as positive mental states.

Anxiety, shock, emaciation, impatience, confusion, stress, tension, responsibility, fear, dissatisfaction, resentment, disgust, shame, contempt, jealousy, guilt, inferiority, grudge, agony, sadness, anger, trouble, resignation, despair, hatred, emptiness, a feeling of wanting to escape, and refusal are classified as negative mental states.

Needless to say, a certain mental state may be classified as both a positive mental state and a negative mental state. For example, stress may be classified as a positive mental state or, conversely, a negative mental state, depending on a scene or a situation where the stress occurs. Therefore, a certain mental state may be classified as either a positive or negative mental state, depending on a scene or a situation where the mental state occurs. Needless to say, each mental state may be classified in advance as either a positive or negative mental state.

Note that a person's state may be the person's state (such as a physical state) other than a mental state. For example, the category of the concept of physical state includes a sense of exhaustion, a sense of fatigue, and the like.

Solution management information is information for identifying a solution which is means for changing a negative mental state to a positive mental state. In short, a solution is means for allowing biological information indicating a positive mental state to be measured from a person from whom biological information indicating a negative mental state is measured. Hereinafter, a person from whom biological information indicating a negative mental state is measured will be referred to as a “specific person”.

A solution is a process of giving a change to at least one of a plurality of persons having a conversation. For example, a solution is a process that acts on at least one of a specific person and a person other than the specific person among a plurality of persons. A person other than a specific person may be a person with the cause of measurement of biological information indicating a negative mental state from the specific person, or a third person other than the person with the cause. The third person may be a person who has a specific relationship with the person with the cause.

For example, a plurality of solutions are registered in the solution management information. To give specific example, a solution is a process of changing the topic of a conversation, a process of directing a conversation to a device other than a person, or a process of separating a specific person from the place where a conversation is taking place. Other solutions may be registered in the solution management information.

For each conversation scene or situation, one or more solutions according to the conversation scene or situation may be determined in advance. For example, for each conversation scene or situation, information indicating the conversation scene or situation and information indicating a solution for changing a negative mental state to a positive mental state in the conversation scene or situation are linked in advance with each other and registered in the solution management information. For example, one or more solutions according to each conversation scene or situation may be determined in advance by using a statistical method.

The processor 24 is configured to obtain biological information of each of a plurality of persons having a conversation, and, in the case where there is, among the plurality of persons, a specific person from whom biological information indicating a negative mental state is measured, control execution of a process of giving a change to at least one of the plurality of persons. The process is the above-described solution.

A solution may be executed by the information processing apparatus 10, may be executed by one or more devices other than the information processing apparatus 10, or may be executed by the information processing apparatus 10 and one or more devices. For example, the information processing apparatus 10 and one or more devices may cooperate to execute a solution, or a plurality of devices may cooperate to execute a solution.

In addition, the processor 24 is configured to control the operation of each unit of the information processing apparatus 10. The processor 24 may include memory.

Note that a process performed by the processor 24 may be executed by an apparatus other than the information processing apparatus 10 (such as a terminal apparatus, a server, or another device). In addition, a part of a process may be executed by the information processing apparatus 10, and another part of the process may be executed by an apparatus other than the information processing apparatus 10. For example, an analysis of biological information (such as a process of estimating a mental state) may be executed by an apparatus other than the information processing apparatus 10 (such as a server), and a solution may be executed by the information processing apparatus 10. Even in this case, a solution may be executed by a device other than the information processing apparatus 10. In addition, information indicating the result of the process may be transmitted from this other apparatus to the information processing apparatus 10 to be displayed on the display device of the UI 16, or may be output as audio information.

Hereinafter, the flow of a process performed by the information processing apparatus 10 will be described with reference to FIG. 3. FIG. 3 is a flowchart illustrating the flow of the process.

The processor 24 obtains biological information of each of a plurality of persons having a conversation (S01). Hereinafter, the processing in step S01 will be described in detail.

For example, image data representing a plurality of persons having a conversation is generated by capturing an image of the plurality of persons with the camera 18. An image of the plurality of persons may be collectively captured with the camera 18, or an image of each of the plurality of persons may be separately captured with the camera 18. In addition, audio data representing the conversation is generated by collecting the conversation with the microphone 20. The processor 24 identifies each of the plurality of persons on the basis of image data representing the plurality of persons and audio data representing the conversation, and estimates the details of the conversation. Technology of the related art (such as face recognition technology and voice recognition technology) may be used as technology for identifying each person from image data and audio data, and technology for estimating the details of a conversation from image data and audio data. For example, the processor 24 identifies each person's personal information (such as sex, age, and name) on the basis of image data and audio data. The processor 24 may use a database where personal information is registered (such as a database where an image representing each person's face, each person's voice, and the like are recorded) may be used for identifying personal information. In addition, the processor 24 may estimate who is talking to whom on the basis of image data and audio data. Note that personal information may be input by each person to the information processing apparatus 10.

A conversation may be a conversation held by a plurality of persons gathering at the same place (such as the same room, venue, or square), a conversation held via a telephone line, a conversation held via a communication channel such as the Internet (such as a chat or a teleconference), or other conversations.

In addition, biological information of each of a plurality of persons having a conversation is measured by the biological information measurement apparatus 12. For example, the biological information measurement apparatus 12 is worn by each of the plurality of persons to measure biological information from each person. Each person's biological information is transmitted from a corresponding biological information measurement apparatus 12 to the information processing apparatus 10, and the processor 24 obtains each person's biological information transmitted from a corresponding biological information measurement apparatus 12.

Next, for each person, the processor 24 estimates the person's mental state on the basis of the person's biological information (S02). Hereinafter, the processing in step S02 will be described in detail.

For each person, the processor 24 compares the person's biological information and each item of reference biological information registered in the above-mentioned state management information, and searches for reference biological information whose difference from the biological information is included in an allowable range. The processor 24 estimates a mental state indicated by state information linked with the reference biological information found by the search as the mental state of the person from whom the biological information is measured.

Note that, in the case where a plurality of items of reference biological information whose differences from the person's biological information are included in the allowable range are found by the search, the processor 24 identifies reference biological information whose difference from the biological information is minimum from among the plurality of items of reference biological information.

Reference biological information may be information indicating a characteristic component of biological information. In this case, the processor 24 may extract a characteristic component from a person's biological information, and may search for reference biological information whose difference from the extracted component is included in an allowable range.

For example, in the case where brain waves are used as biological information, the processor 24 may extract a characteristic component from the measured brain waves and analyze the component to estimate the person's mental state.

In order to analyze a user's brain activity (such as brain waves), a fast-Fourier transform (FFT), a wavelet transform (WT), a time-frequency distribution (TFD), eigenvector methods (EM), or an auto-regressive model (ARM) may be used.

The processor 24 estimates each person's mental state and determines whether there is, among the plurality of persons having a conversation, a specific person with a negative mental state.

In the case where there is, among the plurality of persons having a conversation, no specific person with a negative mental state (NO in S03), the process returns to step S01.

In the case where there is, among the plurality of persons having a conversation, a specific person with a negative mental state (YES in S03), the processor 24 identifies a solution for changing the mental state of the specific person with a negative mental state to a positive mental state by referring to the above-mentioned solution management information (S04). The processor 24 may identify one or more solutions according to the conversation scene or situation. For example, the processor 24 estimates the conversation scene or situation on the basis of image data and audio data.

Next, the processor 24 controls execution of one or more solutions identified in step S04 (S05).

The processor 24 may execute the solution(s) by controlling the information processing apparatus 10, may execute the solution(s) by controlling one or more devices other than the information processing apparatus 10, or may execute the solution(s) by controlling the information processing apparatus 10 and one or more devices. In the case where a negative mental state may not be changed to a positive mental state only with the information processing apparatus 10, the information processing apparatus 10 and one or more devices may cooperate to execute the solution(s), or a plurality of devices may cooperate to execute the solution(s).

The processor 24 obtains biological information of a specific person with a negative mental state, and estimates the mental state of the specific person on the basis of the biological information. For example, the processor 24 estimates whether the specific person has a positive mental state.

The processor 24 may control execution of the solution(s) until biological information indicating a positive mental state is measured from the specific person. In doing so, the solution(s) is/are executed until biological information indicating a positive mental state is measured from the specific person. The processor 24 may execute the solution(s) for a predetermined time period or longer until biological information indicating a positive mental state is continuously measured from the specific person. In the case where biological information indicating a positive mental state is continuously measured from the specific person for the predetermined time period or longer, the processor 24 may stop execution of the solution(s).

In the case where biological information indicating a positive mental state is not measured from the specific person at a time point at which a predetermined time period has elapsed since a time point at which a solution was executed, the processor 24 may change the solution to execute, or may additionally execute another solution.

In the case where biological information indicating a negative mental state is measured from the specific person for a predetermined time period or longer, the processor 24 may control execution of the solution(s); and, in the case where biological information indicating a negative mental state is not measured from the specific person for the predetermined time period or longer, the processor 24 may not control execution of the solution(s). In doing so, in the case where biological information indicating a negative mental state is measured from the specific person for the predetermined time period or longer, the solution(s) is/are executed by the information processing apparatus 10 or another apparatus; and, in the case where biological information indicating a negative mental state is not measured from the specific person for the predetermined time period or longer, the solution(s) is/are not executed.

Each of the above-described time periods may be changed by a person such as the administrator, or may be determined for each person.

Note that the entire processing from step S01 to step S05 described above may be executed by the information processing apparatus 10, or a part of the processing from step S01 to step S05 may be executed by the information processing apparatus 10, and another part of the processing may be executed by an apparatus other than the information processing apparatus 10 (such as a server). In the case where the individual processes are executed by separate apparatuses, various types of data are transmitted and received among a plurality of apparatuses executing the processes.

For example, the process to “obtain biological information” in step S01 and the process to “control execution of the solution(s)” in step S05 may be executed by the information processing apparatus 10, and the process to “estimate the mental state” in step S02 and the process to “identify a solution(s)” in step S04 may be executed by a server or the like other than the information processing apparatus 10. As another example, the processing in step S02 may be executed by a server or the like other than the information processing apparatus 10, and the processing in step S04 may be executed by the information processing apparatus 10.

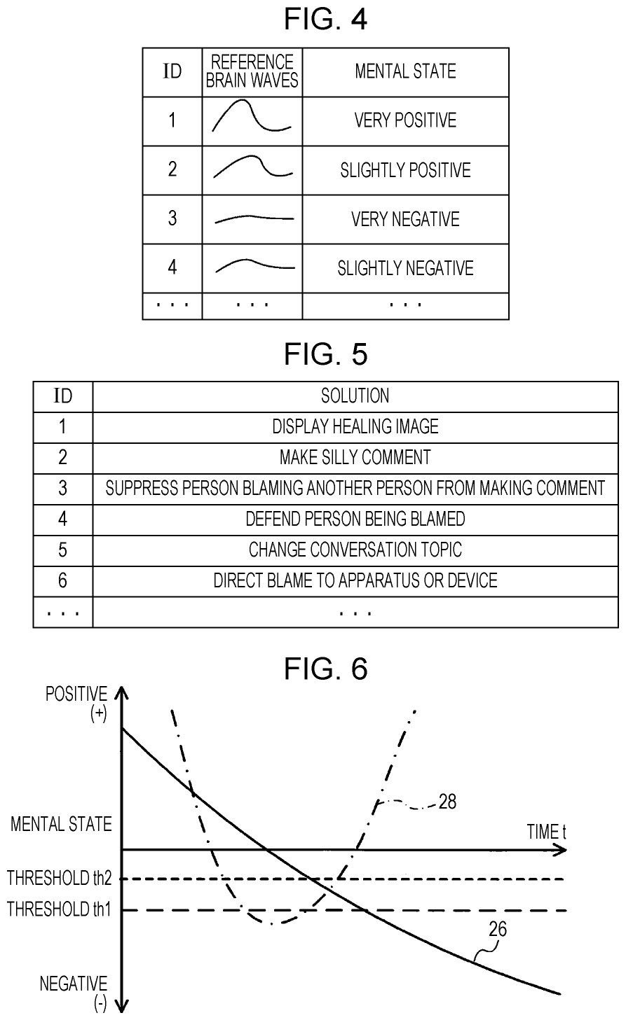

Hereinafter, a specific example of the state management information will be described with reference to FIG. 4. FIG. 4 illustrates an example of a state management table, which is an example of the state management information. Data of the state management table is stored in the storage device 22. Data of the state management table may be stored in another apparatus other than the information processing apparatus 10 (such as a server), instead of being stored in the storage device 22.

In the state management table, an identification (ID), reference brain waves, and state information indicating a person's mental state are linked in advance with one another.

An ID is information for managing each item of information registered in the state management table.

Reference brain waves are one example of reference biological information. Reference brain waves are determined by, for example, statistical processing, and are brain waves that are estimated to be generated from a user who has a mental state indicated by state information linked with the reference brain waves. Although brain waves are used as one example of biological information, biological information other than brain waves may be used.

In FIG. 4, a very positive mental state, a slightly positive mental state, a very negative mental state, and a slightly negative mental state are indicated as examples of a mental state. These are only exemplary, and mental states may be classified in more detail and registered in the state management table.

For example, mental states classified as positive mental states, such as relief, appreciation, and relaxation, and mental states classified as negative mental states, such as anxiety, stress, and agony, may be registered in the state management table. In this case, for each mental state, state information indicating the mental state and reference brain waves estimated to be generated from a person with the mental state are linked with each other and registered in the state management table.

The level of a mental state, such as the level of being positive or the level of being negative, may be registered in the state management table.

For example, even a mental state that is classified as a positive mental state may vary in its level depending on the degree of being positive. For example, a mental state such as appreciation, excitement, or happiness is estimated as a very positive mental state, and a mental state such as relaxation is estimated as a slightly positive mental state. In short, a mental state such as appreciation, excitement, or happiness is estimated to have a greater degree of being positive than a mental state such as relaxation, and is estimated as a mental state with a high level of being positive.

Similarly, even a mental state that is classified as a negative mental state may vary in its level depending on the degree of being negative. For example, a mental state such as agony, fear, or a feeling of wanting to escape is estimated as a very negative mental state, and a mental state such as anxiety or tension is estimated as a slightly negative mental state. In short, a mental state such as agony, fear, or a feeling of wanting to escape is estimated to have a greater degree of being negative than a mental state such as anxiety or tension, and is estimated as a mental state with a high level of being negative.

Note that the level of being positive and the level of being negative may be represented by numerals or the like.

The above classifications are only exemplary, and classifications and levels different from the above classifications may be determined, depending on how mental states are defined.

To give a specific example, brain waves with the ID “1” are brain waves indicating a very positive mental state. Brain waves with the ID “3” are brain waves indicating a very negative mental state.

For example, in the case where brain waves whose difference from reference brain waves with the ID “1” is included in an allowable range is measured from a person, the processor 24 estimates a “very positive mental state” linked with reference brain waves with the ID “1” as the person's mental state.

In the case where brain waves whose difference from reference brain waves with the ID “3” is included in an allowable range is measured from a person, the processor 24 estimates a “very negative mental state” linked with reference brain waves with the ID “3” as the person's mental state.

The processor 24 may calculate the degree of similarity between brain waves measured from a person and reference brain waves and determine whether that degree of similarity is greater than or equal to a threshold. The threshold is a value corresponding to an allowable range. In the case where the degree of similarity between measured brain waves and reference brain waves is greater than or equal to the threshold, the processor 24 determines that the measured brain waves and the reference brain waves are similar. In short, the processor 24 determines that the difference between the measured brain waves and the reference brain waves is included in the allowable range. For example, in the case where brain waves whose degree of similarity with reference brain waves with the ID “1” is measured from a person, the processor 24 estimates a “very positive mental state” as the person's mental state.

For each person, reference biological information and state information may be linked with each other and registered in the state management table. For example, biological information actually measured from a person is registered as the person's reference biological information in the state management table. In addition, state information indicating the person's mental state at the time the biological information was measured is linked with the reference biological information and is registered in the state management table. For example, a person who performs a registration work asks a person about a mental state that the person had when biological information was measured, and registers information indicating this mental state as state information in the state management table by linking the state information with the biological information which is reference biological information.

Although a mental state is estimated on the basis of brain waves in the example illustrated in FIG. 4, a mental state may be estimated on the basis of other biological information. For example, the level of stress that a person feels may be measured by analyzing salivary amylase activity contained in the person's saliva. Alternatively, the level of stress that a person feels may be measured using other stress markers. On the basis of the level of stress that a person feels, the processor 24 may estimate whether the person is having either a positive or negative mental state, or estimate the level of being positive or negative.

In addition, the processor 24 may estimate a person's mental state on the basis of a plurality of types of biological information. For example, the processor 24 may estimate a person's mental state on the basis of brain waves and the volume of saliva. Needless to say, other biological information may be used.

Hereinafter, a specific example of the solution management information will be described with reference to FIG. 5. FIG. 5 illustrates an example of a solution management table, which is an example of the solution management information. Data of the solution management table is stored in the storage device 22. Data of the solution management table may be stored in another apparatus other than the information processing apparatus 10 (such as a server), instead of being stored in the storage device 22.

In the solution management table, an ID and solution information indicating a solution are linked in advance with each other. An ID is information for managing each item of information registered in the solution management table. One or more solutions are registered in the solution management table.

For each conversation detail, scene, or situation, one or more solutions may be determined and registered in the solution management table. For example, information indicating a conversation detail, scene, or situation and solution information indicating a solution estimated to be capable of changing a negative mental state to a positive mental state in the conversation scene or situation are linked in advance with each other and registered in the solution management table. Such solutions are determined in advance.

Specific examples of solutions will be described hereinafter.

The solution with the ID “1” is a process of displaying a healing image. For example, a healing image is an image that has the effect of calming the place where a conversation is taking place. Specifically, a healing image is an image that represents a landscape, an image that represents an animal, or the like. An image that represents a landscape may be an image that represents nature (such as an image that represents sea, mountain, river, or countryside), or an image that represents a building. A healing image may be a still image or a moving image. In addition, the solution with the ID “1” may be a process of generating sound such as music and light, along with displaying a healing image. The processor 24 may display a healing image on the display device of the UI 16, or may display a healing image on an apparatus other than the information processing apparatus 10 (such as a terminal apparatus used by each of persons having a conversation, particularly a terminal apparatus used by a specific person, or another device).

Data of a healing image may be stored in advance in the information processing apparatus 10 or another apparatus (such as a server or a device). In this case, the processor 24 obtains data of a healing image from a device where the data of the image is stored. In addition, the processor 24 may obtain data of a healing image by using the Internet.

In addition, one or more healing images may be determined for each conversation detail, scene, or situation. For example, an image estimated to be capable of changing a negative mental state to a positive mental state is determined in advance for each conversation detail, scene, or situation. For example, the processor 24 analyzes image data generated by capturing an image with the camera 18 and audio data generated by the microphone 20 to estimate the conversation details, scene, or situation, and displays an image in accordance with the conversation details. scene, or situation on the display device. Technology of the related art may be used as the analysis technology and the estimation technology. Note that the processor 24 may analyze either image data generated by capturing an image with the camera 18 or audio data generated by the microphone 20 to estimate the conversation details, scene, or situation. The same applies to the following specific examples.

The solution with the ID “2” is a process of making a silly comment. For example, the processor 24 estimates the details of a conversation on the basis of image data generated by capturing an image with the camera 18 and audio data generated by the microphone 20, and makes a comment unrelated to the details from a speaker. The comment may be a comment directed to a specific person, a comment directed to a person other than a specific person, or a comment directed to all the persons having a conversation.

Data of the comment with the ID “2” may be stored in advance in the information processing apparatus 10 or another apparatus (such as a server or a device). In this case, the processor 24 obtains data of the comment from a device where the data of the comment is stored. In addition, the processor 24 may obtain the data of the comment by using the Internet.

In addition, a comment may be determined for each conversation scene or situation. For example, a comment estimated to be capable of changing a negative mental state to a positive mental state is determined in advance for each conversation scene or situation. For example, the processor 24 analyzes image data generated by capturing an image with the camera 18 and audio data generated by the microphone 20 to estimate the conversation scene or situation, and makes a comment in accordance with the conversation scene or situation.

The solution with the ID “3” is a process of suppressing a person blaming a specific person from making a comment. The processor 24 analyzes image data generated by capturing an image with the camera 18 and audio data generated by the microphone 20 to estimate the conversation details, scene, or situation, and estimates a person blaming a specific person. For example, a process of stopping or discouraging a person blaming a specific person from making a comment is a process of suppressing a person from making a comment.

Data indicating a process of suppressing a person from making a comment is stored in advance in the information processing apparatus 10 or another apparatus (such as a server or a device). The processor 24 obtains information indicating the process from a device where the information is stored. In addition, the processor 24 may obtain the information by using the Internet. The processor 24 executes, for example, a process of stopping or discouraging a person blaming a specific person from making a comment in accordance with the information. To do so, the processor 24 displays an image or makes a comment. In addition, in the case where the information processing apparatus 10 is a robot, the processor 24 may control the movement of the information processing apparatus 10, which is a robot. For example, the information processing apparatus 10 may move to a position where a person blaming a specific person is, and may stop the person from making a comment. The processor 24 may stop the person from making a comment by controlling the movement of a robot other than the information processing apparatus 10.

In addition, a method of suppressing a person blaming a specific person from making a comment may be determined for each conversation detail, scene, or situation. For example, a method of suppressing a person from making a comment, which is estimated to be capable of changing a negative mental state to a positive mental state, is determined in advance for each conversation detail, scene, or situation. The processor 24 analyzes image data generated by capturing an image with the camera 18 and audio data generated by the microphone 20 to estimate the conversation details, scene, or situation, and suppresses a person blaming a specific person from making a comment by using a method in accordance with the conversation details, scene, or situation.

The solution with the ID “4” is a process of defending a specific person being blamed. For example, data of a comment for defending a specific person is stored in advance in the information processing apparatus 10 or another apparatus (such as a server or a device). The processor 24 obtains data of the details of the comment from a device where the data of the comment is stored. The processor 24 may obtain the data of the comment by using the Internet.

In addition, a method of defending a specific person may be determined for each conversation detail, scene, or situation. For example, a defending method estimated to be capable of changing a negative mental state to a positive mental state is determined in advance for each conversation detail, scene, or situation. The processor 24 analyzes image data generated by capturing an image with the camera 18 and audio data generated by the microphone 20 to estimate the conversation details, scene, or situation, and defends a specific person by using a method in accordance with the conversation details, scene, or situation.

The solution with the ID “5” is a process of changing the topic of a conversation. The processor 24 analyzes image data generated by capturing an image with the camera 18 and audio data generated by the microphone 20 to estimate the details of a conversation, and makes a comment on a topic different from the topic of the conversation from a speaker. For example, for each conversation topic, a topic different from the conversation topic is determined, and information indicating the different topic is stored in advance in the information processing apparatus 10 or another apparatus (such as a server or a device). The processor 24 obtains information indicating the different topic from a device where the information is stored. The processor 24 may obtain the information by using the Internet.

In addition, for each conversation scene or situation, a topic different from the topic of the conversation may be determined. The processor 24 analyzes image data generated by capturing an image with the camera 18 and audio data generated by the microphone 20 to estimate the conversation scene or situation, and changes the conversation topic in accordance with the conversation scene or situation.

The solution with the ID “6” is a process of, in the case where a specific person is being blamed, directing the blame to an apparatus (such as the information processing apparatus 10 or a device) other than the specific person. A process of directing blame to an apparatus other than a specific person includes, for example, a process of distracting a person blaming a specific person (such as a process of emitting sound or light), a process of making a comment to offend a person blaming a specific person, a process of making a comment on a matter estimated to be of interest to a person blaming a specific person, a process of displaying an image that offends a person blaming a specific person, or a process of displaying an image that represents a matter of interest to a person blaming a specific person. For example such a comment or an image is determined in advance, and data of the comment or data of the image is stored in advance in the information processing apparatus 10 or another apparatus (such as a server or a device). The processor 24 obtains data of the comment or data of the image from a device where the data of the comment or the data of the image is stored. The processor 24 may obtain the data of the comment or the data of the image by using the Internet.

In addition, a process of directing blame to an apparatus may be determined for each conversation detail, scene, or situation. The processor 24 analyzes image data generated by capturing an image with the camera 18 and audio data generated by the microphone 20 to estimate the conversation details, scene, or situation, and performs a process of directing blame to an apparatus in accordance with the conversation details, scene, or situation.

The solutions illustrated in FIG. 5 are only exemplary, and solutions other than those illustrated in FIG. 5 may be executed. For example, the processor 24 may execute a process of distracting persons having a conversation (such as a process of emitting sound or light).

Hereinafter, an exemplary process performed by the information processing apparatus 10 will be described with reference to FIG. 6. FIG. 6 is a graph illustrating changes in mental state over time. Time is plotted on the abscissa, and the level of a mental state is plotted on the ordinate. The positive side indicates a positive mental state, and the negative side indicates a negative mental state. The higher the level on the positive side (that is, the greater the absolute value on the positive side), the higher the level of being positive. The higher the level on the negative side (that is, the greater the absolute value on the negative side), the higher the level of being negative.

For example, as indicated by reference numeral 26, in the case where biological information whose level of being negative tends to be higher over time is measured from a specific person, the processor 24 may execute a solution. In short, in the case where the degree of being negative becomes higher over time, the processor 24 controls execution of a solution in order to prevent the mental state of a specific person from becoming more negative.

In the case where biological information indicating a negative mental state is measured from a specific person and then biological information indicating a positive mental state is measured from the specific person within a predetermined time period, the processor 24 need not execute a solution. For example, a mental state indicated by reference numeral 28 indicates a negative mental state at first, and then the level of being negative becomes higher over time. However, after that, the mental state changes to a positive mental state, and the level of being positive becomes higher over time. For example, in the case where biological information indicating a positive mental state is measured from a specific person within a predetermined time period from a time point at which biological information indicating a negative mental state was measured from the specific person, the processor 24 does not execute a solution.

For example, even in the case where a specific person is scolded by his/her boss or senior colleague, if the mode of the scolding is appropriate and the specific person has a feeling of appreciation, biological information indicating a positive mental state may be measured from the specific person. For example, even in the case where the specific person has a negative mental state at the beginning of the scolding, after that, the specific person may have a positive mental state. In such a case, there is no need to execute a solution, and the processor 24 does not execute a solution.

In contrast, in the case where the mode of the scolding is inappropriate and, as indicated by reference numeral 26, the level of being negative becomes higher over time, the processor 24 executes a solution.

The processor 24 executes a solution in the case where the level of being negative becomes greater than or equal to a threshold th1; and the processor 24 need not execute a solution in the case where the level of being negative is less than the threshold th1. The fact that the level of being negative becomes greater than or equal to the threshold th1 means that the absolute value on the negative side becomes greater than or equal to the threshold th1.

For example, because the mental state indicated by reference numeral 26 is such that the level of being negative is greater than or equal to the threshold th1, the processor 24 executes a solution.

In the case where biological information of a specific person indicates a negative mental state whose level is greater than or equal to the threshold th1 for a predetermined time period or longer from a time point at which the level of being negative became greater than or equal to the threshold th1, the processor 24 may execute a solution. Otherwise, the processor 24 does not execute a solution.

The mental state indicated by reference numeral 26 is such that biological information indicating a negative mental state whose level is greater than or equal to the threshold th1 is measured from a specific person for a predetermined time period or longer from a time point at which the level of being negative became greater than or equal to the threshold th1. In this case, the processor 24 executes a solution.

The mental state indicated by reference numeral 28 is such that, although the level of being negative is greater than or equal to the threshold th1, biological information indicating a negative mental state whose level is greater than or equal to the threshold th1 is not measured from a specific person for a predetermined time period or longer from a time point at which the level of being negative became greater than or equal to the threshold th1. In this case, the processor 24 does not execute a solution.

The processor 24 may change a condition for executing a solution in accordance with a person's condition. A condition may be a mental condition (such as a mental state) or a physical condition (such as physical fatigue). The processor 24 may estimate a person's condition on the basis of the person's biological information. As another example, each person may input information indicating the person's condition to the information processing apparatus 10. In addition, each person may input information indicating the person's condition to the person's terminal apparatus, and the information processing apparatus 10 may obtain information indicating the person's condition from the person's terminal apparatus.

Changing a condition for executing a solution is, for example, changing a negative level threshold.

The threshold th1 is a threshold used for a person estimated to be in good condition. A threshold th2 is a threshold used for a person estimated to be in bad condition. The threshold th2 is a value shifted to the positive side (that is, a value with a lower level of being negative) than the threshold th1.

By using the threshold th2 for a person in bad condition, compared with the case of using the threshold th1, the processor 24 executes a solution even in the case where the level of being negative is lower. In short, a solution is executed for a person in bad condition, compared with a person in good condition, even in the case where the level of being negative is lower.

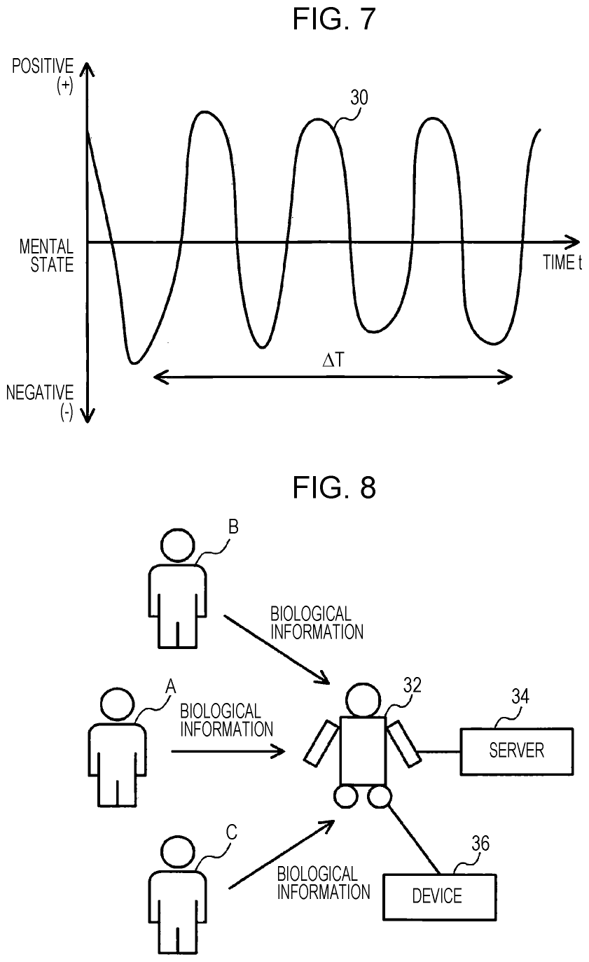

Hereinafter, another mental state will be described with reference to FIG. 7. FIG. 7 is a graph illustrating changes in mental state over time. The definition of the abscissa and the ordinate is the same as that of the graph illustrated in FIG. 6.

As indicated by reference numeral 30, in the case where biological information indicating a negative mental state and biological information indicating a positive mental state are alternately measured from a specific person within a predetermined time period ΔT, the processor 24 may execute a solution. As described here, the processor 24 executes a solution in the case where the mental state is unstable. For example, even in the case where biological information indicating a negative mental state is measured from a specific person and then biological information indicating a positive mental state is measured from the specific person, if biological information indicating a negative mental state is measured from the specific person after that, the specific person is estimated to have an unstable mental state. In this case, the processor 24 executes a solution.

Hereinafter, referring to FIG. 8, a specific example will be described. FIG. 8 schematically illustrates persons, a robot, and so forth.

Here, as an example, it is assumed that persons A, B, and C are having a conversation. A robot 32 is, for example, a self-propelled apparatus, and is an apparatus capable of having a conversation or performing a task, for example. In addition, the robot 32 is an apparatus that includes the information processing apparatus 10 and the biological information measurement apparatus 12. In short, the robot 32 includes the processor 24, and has the function of executing processing of the above-described information processing apparatus 10.

A server 34 may be used. The entirety or part of processing of the information processing apparatus 10 may be executed by the server 34. In this case, processing executed by the server 34 is not executed by the robot 32. For example, an analysis of biological information may be executed by the server 34, and control of execution of a solution may be executed by the robot 32. A plurality of servers 34 may be used.

A device 36 is used for realizing a solution. The device 36 may not be used, depending on a solution. A plurality of devices 36 may be used.

The device 36 is an apparatus such as a personal computer (PC), a tablet PC, a smartphone, a cellular phone, a robot (such as a humanoid robot, a non-human animal robot, a cleaning robot, and other robots), a projector, a display device such as a liquid crystal display, a recording device, a reproducing device, an image capturing device such as a camera, a refrigerator, a rice cooker, a microwave oven, a coffee maker, a vacuum cleaner, a washing machine, an air conditioner, lighting equipment, health equipment, a clock, a monitoring camera, an automobile, a two-wheeled vehicle, an aircraft (such as an unmanned aircraft (e.g., a drone)), a game machine, a gas range, a warm water washing toilet seat, a ventilation fan, a doorbell, an entrance monitor, an elevator, a door, a window, or various sensing devices (such as a temperature sensor, a humidity sensor, a voltage sensor, and a current sensor). The category of the concept of the device 36 may include devices in general. For example, an information device, a video device, an audio device, and other devices may be included in the category of the concept of the device 36 according to the present exemplary embodiment. The device 36 includes, for example, a communication device which is a communication interface, a storage device that stores data, and a processor that controls the operation of the device 36. The device 36 may include a user interface. The device 36 may transmit device identification information for identifying the device 36, which is a local device, to the information processing apparatus 10. Device identification information includes, for example, the ID, name, model number, or address (such as media access control address (MAC address) or Internet Protocol address (IP address)) of the device 36.

For example, the robot 32, the server 34, and the device 36 may communicate with one another via a communication channel such as a network, or may directly communicate with one another using short-range wireless communication or the like.

The robot 32 measures biological information of each of the persons A, B, and C, and analyzes each person's biological information, thereby estimating each person's mental state. For example, the state management table illustrated in FIG. 4 is used for the estimation.

In addition, the robot 32 analyzes image data generated by capturing an image with the camera 18 and audio data generated by the microphone 20 to identify the persons A, B, and C, and estimates the conversation details, scene, or situation. Note that each of the persons A, B, and C may input his/her personal information to the robot 32.

For example, in the case where biological information indicating a negative mental state is measured from the person A when the persons A, B, and C are having a conversation, the robot 32 executes a solution to allow biological information indicating a positive mental state to be measured from the person A.

For example, the robot 32 executes a solution so that the person A will not be the target of the conversation. Specifically, the robot 32 performs a process of making a comment (such as making a comment to discourage a person (such as the person B) blaming the person A), emitting sound or light, changing the conversation topic, or directing the blame to the robot 32 or the device 36. Other solutions may be executed.

In addition, the robot 32 may execute a solution that acts on the person A, a solution that acts on the person B blaming the person A, a solution that acts on both the persons A and B, or a solution that acts on the person C other than the persons A and B. For example, a solution that acts on the person A is a process of defending the person A, and a solution that acts on the person B is a process of discouraging the person B. For example, the robot 32 analyzes image data generated by capturing an image with the camera 18 and audio data generated by the microphone 20 to identify the person B blaming the person A or the person C other than the persons A and B.

The robot 32 may record a conversation history, and change the conversation topic on the basis of the history to allow the person A to have a positive mental state. For example, the robot 32 records the topic of a conversation when biological information indicating a positive mental state is measured from the person A, and changes the conversation topic to the recorded topic when biological information indicating a negative mental state is measured from the person A.

In a situation where the person B is blaming the person A, if biological information indicating regret is measured from the person A, the robot 32 need not execute a solution. Also in this case, if biological information whose level of being negative is greater than or equal to a threshold th (such as the threshold th1 or the threshold th2) is measured from the person A, the processor 24 may execute a solution. In addition, the robot 32 may paraphrase the details of a conversation held by the person A and the person B. For example, the robot 32 may paraphrase an offensive tone into a non-offensive tone (such as mild expressions).

The robot 32 may change a condition for executing a solution in accordance with a specific person's attribute. A person's attribute includes, for example, age, sex, and the position or rank in an organization such as a company.

Changing a condition for executing a solution is, for example, changing the threshold illustrated in FIG. 6. For example, in the case where the age of the person A, who is a specific person, is older than or equal to a threshold, the processor 24 may determine whether to execute a solution by using the threshold th1; and, in the case where the age of the person A is younger than the threshold, the processor 24 may determine whether to execute a solution by using the threshold th2. In addition, in the case where the position of the person A in an organization is greater than or equal to a predetermined position, the processor 24 may determine whether to execute a solution by using the threshold th1; and, in the case where the position of the person A is less than the predetermined position, the processor 24 may determine whether to execute a solution by using the threshold th2. The same applies to sex and the like.

In addition, the robot 32 may change a condition for executing a solution in accordance with the difference between the attribute of a specific person and the attribute of a person with the cause of measurement of biological information indicating a negative mental state from the specific person.

For example, the person A is a specific person whose biological information indicating a negative mental state is identified. The person B is a person blaming the person A. Because the person B is blaming the person A, biological information indicating a negative mental state is measured from the person A. In short, the person B is a person with the cause of measurement of biological information indicating a negative mental state from the person A. The robot 32 analyzes image data generated by capturing an image with the camera 18 and audio data generated by the microphone 20 to identify the person B blaming the person A.

For example, the robot 32 executes a solution in the case where the difference between the age of the person A and the age of the person B is less than a threshold. In the case where the persons A and B are from the same generation or of the same age, the problem may not be solved by the parties concerned; thus, the robot 32 executes a solution.

The robot 32 does not execute a solution in the case where the difference between the age of the person A and the age of the person B is greater than or equal to the threshold. Because such a case may be the case where the person B is instructing the person A, the robot 32 does not execute a solution.

The robot 32 need not execute a solution in the case where the person B is older than the person A and the difference between the age of the person A and the age of the person B is greater than or equal to the threshold. Because such a case may be the case where the person B is a boss and the person A is a subordinate, it is conceivable that the problem may be solved by the parties concerned. Therefore, the robot 32 does not execute a solution.

The robot 32 may prompt a third person that is other than the person A, who is a specific person, and that is other than the person B, who is a person with the cause of measurement of biological information indicating a negative mental state from the person A, to execute a solution. For example, the robot 32 prompts the person C, who is a third person, to execute a solution. The robot 32 analyzes image data generated by capturing an image with the camera 18 and audio data generated by the microphone 20 to identify the person C, who is a third person. For example, the robot 32 may prompt the person C to stop the person B from blaming the person A, prompt the person C to change the conversation topic, or prompt the person C to defend the person A. The robot 32 may display a message prompting execution of a solution on the display device, emit sound expressing the message, or move to the person C to prompt the person C to execute a solution.

The above-mentioned third person may be, for example, a person who has a specific relationship with a person with the cause of measurement of biological information indicating a negative mental state from a specific person. For example, the third person may be a boss or a senior colleague of the person with the cause, or a person older than the person with the cause. In short, the third person may be a person estimated to be capable of stopping or suppressing actions taken by the person with the cause.

In addition, a solution may be a process of directing a comment made by the person B, who is a person with the cause, to an apparatus (such as the robot 32 or the device 36) capable of having a conversation with a person. For example, the robot 32 talks to the person B (such as calling the name of the person B), makes a comment on a matter of interest to the person B, displays an image regarding the matter on the display device, makes a comment to distract the person B, or makes a comment to offend the person B. The robot 32 may emit directional sound to the person B.

In the case where the number of specific persons is greater than or equal to a threshold, the robot 32 executes a solution; and, in the case where the number of specific persons is less than the threshold, the robot 32 need not execute a solution. For example, in the case where a plurality of persons are being blamed, the number of persons from whom biological information indicating a negative mental state is measured may be greater than or equal to the threshold.

In the case where there are a plurality of persons with the cause of measurement of biological information indicating a negative mental state from a specific person, a solution may be a process that acts on the plurality of persons. For example, in the case where one or more specific persons are being blamed by a plurality of persons, the robot 32 executes a process of stopping the plurality of blaming persons from making a comment.

In addition, in the case where the number of specific persons is less than the threshold and there are a plurality of persons with the cause, a solution may be a process that acts on the plurality of persons with the cause. In the case where the number of persons with the cause is greater than the number of specific persons, a solution may be a process that acts on the plurality of persons with the cause.

The robot 32 may execute a process of separating a specific person (such as the person A) from the place where a conversation is taking place. For example, even in the case where the topic of a conversation is changed or a person with the cause is stopped from making a comment, if biological information indicating a positive mental state is not measured from a specific person, the robot 32 executes a process of separating the specific person from the place where the conversation is taking place. The robot 32 may move by itself and forcibly take the specific person out, or may output sound or display a message to allow the specific person to escape from the place. For example, the robot 32 may display a message indicating that a boss, a friend, or the like is calling the specific person, or may output sound expressing the message.

In addition, the robot 32 may separate a specific person (such as the person A) from a person with the cause (such as the person B) so that the difference between the position of the specific person and the position of the person with the cause becomes greater than or equal to a threshold. For example, the robot 32 may obtain each person's position information by using the Global Positioning System (GPS) and the camera 18 or another camera, and identify the positional relationship between the persons on the basis of each person's position information. It is conceivable that the specific person may be relieved by allowing the specific person to escape to a place where the difference between the position of the specific person and the position of the person with the cause becomes greater than or equal to a threshold (that is, a place where the distance between the specific person and the person with the cause becomes greater than or equal to a threshold).

In the embodiment above, the term “processor” refers to hardware in a broad sense. Examples of the processor includes general processors (e.g., CPU: Central Processing Unit), dedicated processors (e.g., GPU: Graphics Processing Unit, ASIC: Application Integrated Circuit, FPGA: Field Programmable Gate Array, and programmable logic device).

In the embodiment above, the term “processor” is broad enough to encompass one processor or plural processors in collaboration which are located physically apart from each other but may work cooperatively. The order of operations of the processor is not limited to one described in the embodiment above, and may be changed.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus comprising:
a processor configured to:
obtain biological information of each of a plurality of persons having a conversation; and
in a case where there is, among the plurality of persons, a specific person from whom biological information indicating a negative mental state is measured, control execution of a process of giving a change to at least one of the plurality of persons.

2. The information processing apparatus according to claim 1, wherein the processor controls execution of the process until biological information indicating a positive mental state is measured from the specific person.

3. The information processing apparatus according to claim 1, wherein the processor controls execution of the process in a case where biological information indicating a negative mental state is measured from the specific person for a predetermined time period or longer.

4. The information processing apparatus according to claim 2, wherein the processor controls execution of the process in a case where biological information indicating a negative mental state is measured from the specific person for a predetermined time period or longer.

5. The information processing apparatus according to claim 1, wherein the processor controls execution of the process in a case where biological information whose level of being negative tends to be higher over time is measured from the specific person.

6. The information processing apparatus according to claim 1, wherein the processor does not execute the process in a case where biological information indicating a negative mental state is measured from the specific person and then biological information indicating a positive mental state is measured from the specific person within a predetermined time period.

7. The information processing apparatus according to claim 1, wherein the processor controls execution of the process in a case where biological information indicating a negative mental state and biological information indicating a positive mental state are alternately measured from the specific person within a predetermined time period.

8. The information processing apparatus according to claim 1, wherein the processor changes a condition for executing the process in accordance with a condition of the specific person.

9. The information processing apparatus according to claim 1, wherein the processor changes a condition for executing the process in accordance with an attribute of the specific person.

10. The information processing apparatus according to claim 9, wherein the processor changes a condition for executing the process in accordance with a difference between the attribute of the specific person and the attribute of a person with a cause of measurement of biological information indicating a negative mental state from the specific person.

11. The information processing apparatus according to claim 1, wherein the process is a process that acts on at least one of the specific person, and a person other than the specific person among the plurality of persons.

12. The information processing apparatus according to claim 1, wherein the processor prompts a third person who is other than the specific person and who is other than a person with a cause of measurement of biological information indicating a negative mental state from the specific person to execute the process.

13. The information processing apparatus according to claim 12, wherein the third person is a person who has a specific relationship with the person with the cause of measurement of biological information indicating a negative mental state from the specific person.

14. The information processing apparatus according to claim 1, wherein the process is a process of changing a topic of the conversation.

15. The information processing apparatus according to claim 1, wherein the process is a process of directing a comment made by a person with a cause of measurement of biological information indicating a negative mental state from the specific person to an apparatus capable of having a conversation with a person.

16. The information processing apparatus according to claim 1, wherein the processor controls execution of the process in a case where a number of the specific persons is greater than or equal to a threshold.

17. The information processing apparatus according to claim 1, wherein, in a case where there are a plurality of persons with a cause of measurement of biological information indicating a negative mental state from the specific person, the process is a process that acts on the plurality of persons.

18. The information processing apparatus according to claim 1, wherein the process is a process of separating the specific person from a place where the conversation is taking place.

19. The information processing apparatus according to claim 13, wherein the process is a process of separating the specific person from a place where the conversation is taking place.

20. A non-transitory computer readable medium storing a program causing a computer to execute a process, the process comprising:
obtaining biological information of each of a plurality of persons having a conversation; and
in a case where there is, among the plurality of persons, a specific person from whom biological information indicating a negative mental state is measured, controlling execution of a process of giving a change to at least one of the plurality of persons.
